(19) United States
(12) Patent Application Publication
(10) Pub. No.: US 2005/0147578 A1
(43) Pub. Date: Jul. 7, 2005

(54) Topical cosmetic composition having a natural plant active ingredient and method of using same

(75) Inventors: Gopinathan K. Menon, Wayne, NJ (US); Sujatha Dokka, San Marcos, CA (US); Brian Jones, Warwick, NY (US)

Correspondence Address: CHARLES N.J. RUGGIERO, ESQ.;OHLANDT, GREELEY, RUGGIERO & PERLE, L.L.P., 10th FLOOR, ONE LANDMARK SQUARE, STAMFORD, CT, 06901-2682, US

(73) Assignee: Avon Products, Inc.

(21) Appl. No.: 11/059,964

(22) Filed: Feb. 17, 2005

Related U.S. Application Data
(63) Continuation of application No. 10/034,336, filed on Dec. 28, 2001, now abandoned.

(51) Int. Cl. A61K 35/78; A01N 65/00

(52) U.S. Cl. 424/74; 424/727; 424/761

(57) ABSTRACT

There are disclosed topical cosmetic compositions having at least one natural active ingredient and a pharmaceutically or cosmetically acceptable vehicle. There is also a method for improving the aesthetic appearance of skin, hair and/or nails that includes topically applying the compositions of the present invention.

### BACKGROUND OF THE INVENTION

1. Field of the Invention

The present invention relates to topical compositions having an active ingredient derived from a natural plant material. More particularly, the present invention relates to topical compositions that improve the aesthetic appearance of the skin, hair and/or nails. Still more particularly, the present invention relates to methods for using the topical compositions.

2. Description of the Prior Art

Active ingredients derived from plants and plant seeds have commonly been employed in topical compositions for a myriad of medicinal, therapeutic and cosmetic purposes. Such actives can be obtained from various parts of a plant such as seeds, needles, leaves, roots, bark, cones, stems, rhizomes, callus cells, protoplasts, organs and organ systems, and meristems. Active ingredients are incorporated in such compositions in a variety of forms. Such forms include a pure or semi-pure component, a solid or liquid extract or derivative, or a solid plant matter. Plant matter may be incorporated in a variety of subforms such as whole, minced, ground or crushed.

A problem commonly encountered when using an active ingredient derived from a plant and plant seed is the relatively low level at which they are naturally present. Such low levels frequently require relatively large amounts of plant leaf/tissue or seed be processed in order to obtain desired or useful quantities of actives. For rare plants or plant seeds, such large amounts may be unavailable or difficult to obtain.

There is active contemporary interest in the cosmetics industry to develop products that may be applied topically to the skin that provide anti-aging, hydrating, and/or skin texturizing benefits. Cosmetic products that enhance the appearance of skin are increasingly in demand. Consumers are interested in mitigating or delaying the signs of chronologically, hormonally and/or photo-aged skin, such as fine lines, wrinkles, dry skin, and sagging skin. During the aging process, the complexion of the skin, i.e., the color and appearance of the skin, deteriorates slowly from intrinsic aging and/or exposure to sunlight. Cosmetic surgery can be used as a treatment for aged skin, but such treatment is costly and carries the risks normally associated with anesthesia and surgery. Alternatively, cosmetic products that are able to provide anti-aging benefits are highly desirable, to both manufacturers and consumers.

The number of cosmetic products directed toward helping the skin of consumers look younger and less wrinkled is steadily increasing. Commonly, such products contain organic acids as active ingredients. Such anti-aging active ingredients include, for example, α-hydroxy acids (i.e., lactate, glycolate, citrate), β-hydroxy acids (i.e., salicylate; 5-n-octanoylsalicylate) and retinoids (retinoic acids; retinol). It is known that these anti-aging active ingredients have a significant disadvantage in that they frequently are associated with consumer discomfort characterized by burning, smarting, itching or sensation of tightness after application. There remains a general need in the cosmetics industry for products that retard or counter aging effects on the skin, and more specifically for products that produce such effects without undesirable side effects.

More particularly, in view of the previous discussion of demands and limitations in the cosmetics industry, there remains a need for topically applied, cosmetic compositions that have anti-aging and skin texture benefits using natural ingredients as active components.

Plant extracts are commonly used in a variety of herbal compositions having therapeutic uses. For example, U.S. Pat. No. 6,168,795 is directed to an anticancer therapy comprising administering an herbal extract-based composition having an extract of Gynostemma pentaphyllum and other plant extracts. U.S. Pat. No. 5,910,308 to D'Jang is also directed to a herbal extract-based composition comprising an extract of Gynostemma pentaphyllum.

Extracts of Azadirachta indica, the neem tree, as well as other plants in the family Meliaceae, are known to have insecticidal activity. Azadirachtin, a major active ingredient of many of these extracts, is a liminoid of the tetranortriterpenoid type useful in commercial insecticides, which has been shown to be a potent insect growth regulator and feeding deterrent. Methods for producing azadirachin concentrates from neem seed materials are known in the art. U.S. Pat. No. 5,698,423 to Holowach-Keller et al. is directed to a method for producing azadiractin by cell culture of Azadiractin indica.

In spite of the various anti-aging cosmetic products on the market for the treatment of skin, there remains a need for effective topically applied cosmetic compositions that provide anti-aging or rejuvenating benefits to the skin, hair and/or nails using natural ingredients as active components.

### SUMMARY OF THE INVENTION

It is an object of the present invention to provide a topical cosmetic composition that delivers an effective level of an active ingredient from a natural plant material.

It is another object of the present invention to provide topical cosmetic compositions having a natural plant active ingredient or blends of natural plant active ingredients in a pharmaceutically or cosmetically acceptable vehicle.

It is a further object of the present invention to provide a method for topically applying such compositions.

It is still a further object of the present invention to provide a method for delivering a consistent level of an active ingredient to skin, nail and/or hair by topically applying a composition having one or more natural plant active ingredients.

These and other objects and advantages of the present invention, and equivalents thereof, are achieved by cosmetic compositions having a single natural botanical ingredient or blends of natural botanical ingredients, and use of such compositions for topical application. Cosmetic compositions of the present invention have active botanical ingredients from the following group: Gynostemma; Coconut water; Azadirachta; or Rhodeola, and a pharmaceutically or cosmetically acceptable vehicle. Preferably, when compositions of the present invention have Azadirachta as a neem seed cell culture, the compositions also have at least one additional active ingredient selected from the following: Gynostemma, Rhodeola and Coconut water. Additional compositions of the invention have a plant ingredient from the following group: Gynostemma, Coconut water and Rhodeola; and a pharmaceutically or cosmetically acceptable vehicle. Methods of improving the aesthetic appearance of skin, hair and/or nails by topically applying compositions of the invention are provided.

### BRIEF DESCRIPTION OF THE DRAWINGS

FIG. 1 shows the effect of various test samples on the production of heat shock protein in keratinocyte cells in vitro.

FIG. 2 shows the effect of various test samples on apoptosis in keratinocyte cell cultures.

FIG. 3 shows the effect of Gymnostemma on the production of ATP in keratinocyte cell cultures.

FIG. 4 shows the effect of Rhodeola on the production of heat shock protein in keratinocyte cell cultures.

FIG. 5 shows the effect of Coconut water on the production of heat shock protein in keratinocyte cell cultures.

FIG. 6 shows the effect of Azadirachta on the production of heat shock protein in keratinocyte cell cultures.

FIG. 7 shows the effect of Gynostemma on the production of heat shock protein in keratinocyte cell cultures.

### DETAILED DESCRIPTION OF THE INVENTION

The present invention provides topical compositions having a natural plant ingredient or blends of natural plant ingredients that rejuvenate or enhance the skin, hair and/or nails by modifying the response of the cells in such skin, hair and/or nails to stress proteins, in particular heat shock proteins. Compositions of the present invention provide a variety of anti-aging and skin texture benefits.

Improvements in the aesthetic appearance of the skin, hair and/or nails are achieved by topical application of compositions of the present invention. Such compositions have a single natural plant ingredient or a blend of natural plant ingredients.

The plant ingredients preferably include the following: Gynostemma; coconut water; Azadirachta (neem including its cell cultures, broth and/or extracts); and Rhodeola. Especially preferred blends of plant ingredients are Azadirachta and one or more other ingredient, particularly Gynostemma, coconut water and/or Rhodeola. The amount of the active botanical ingredient(s) in the compositions of the present invention is about 0.0001 percentage by weight or weight percent (wt %) to about 50 wt %, preferably about 0.001 wt % to about 10 wt %, more preferably about 0.01 wt % to about 5 wt %, and still more preferably about 0.1 wt % to about 3 wt %, of the total weight of the composition.

Neem is derived from the plant Azadirachta indica of the Meliaceae family. Neem seed cells are especially preferred for use in the present invention because they are prolific and produce a broad range of active ingredients. In the present invention, a neem seed cell is generally obtained by extracting the cells from seed, preferably the seed embryo, and then culturing and homogenizing them in vitro. This process significantly increases the amount and number of seed cells. Preferably, the seed cells are cultured in a medium that promotes the undifferentiated state. More preferably, after homogenizing, the seed cells and the plant cell constituents are preserved. Examples of active ingredients produced by a neem seed cell includes, but are not limited to, 17-beta-hydroxyazadirdione; 17-epiazadiradione; 1alpha-methoxy-1,2-dihydroazadiradione; 1beta,2beta-diepoxy-azadiradione; 22,23-dihydro-23beta-methoxy-azadiradione, 7-desacetyl-gedunin; azadirachtin; azadirone; epoxyazadiradione; meldenin; meliantriol; nimolicinol; and vepinin. Teachings to topical compositions having one or more neem seed cells and neem seed cell broths are shown in co-pending application titled Topical Cosmetic Composition With Skin Rejuvenation Benefits, filed Nov. 9, 2001, which is incorporated herein by reference.

Neem seed cell broth is prepared by tissue culture of cells isolated from neem seed. Neem seed cells are cultured in tissue culture medium containing appropriate nutrients and ingredients. This process provides for rapid cell growth and the production of key active compounds from the neem seed cells. The use of cell bioengineering in the form of plant tissue culture provides a standardized predictably high potency supply of this ingredient of the invention. In vitro studies of the present invention have shown that neem seed cell broth improves cell proliferation of keratinocytes, increases fibroblast metabolism, decreases skin pigmentation, increases pro-collagen synthesis, and binds effectively to estrogen receptor. The preferred amount of neem seed culture/broth or its extracts is about 0.001 wt % to about 10 wt % of the total weight of the composition. Neem seed cell broth has tissue cultured neem seed cells and culture medium.

Gynostemma is a member of the cucumber family. It is also known as 5-Leaf Ginseng, Jiaogulan or Southern Ginseng. It has traditionally been grown in a mountainous region of South Central China. This herb is a different plant from what is commonly known as ginseng. It is a rich source of saponins referred to as “gypenosides”, which are similar, and in some cases identical, to the ginsenosides found in ginseng, but are found at levels several fold higher than those found in ginseng. These saponins have been shown to have antioxidant or cell protective effects. Gynostemma (Jiaogulan) is purchased as a powder form. In cosmetic compositions of the present invention, the preferred amount of Gynostemma is about 0.001 wt % to about 10 wt % based on the total weight of the composition.

Coconut water may be in liquid or powder form, particularly freeze-dried form. In cosmetic compositions of the present invention, the preferred amount of coconut water is about 0.001 wt % to about 10 wt % based on the total weight of the composition. Coconut water is the water that is in the middle of the coconut. As coconut fruit matures, the water “dries up” or is utilized by the plant/fruit.

Rhodeola is purchased as a powder form. In cosmetic compositions of the present invention, the preferred amount of Rhodeola is about 0.001 wt % to about 10 wt % based on the total weight of the composition.

The present invention provides an effective delivery of medicinal, therapeutic and cosmetic active ingredients derived from the above plant sources, including plant seeds. A seed typically has a seed coat and an embryo surrounded by endosperm. The plant seed cells are extracted principally from the tissue of the embryo. Embryonic cells that are located in “non-seed” portion (i.e. rhizomes) of plants are also contemplated for use in the present invention. It is preferred to provide the total (i.e. entire constituency) of the seed cell. The total seed cell is broken down through breaking or fracturing of cell walls to deliver the broad range of the plant cell constituents. The plant seed cells, as well as other plant material or blends of plant materials, are then incorporated into a pharmaceutically or cosmetically acceptable vehicle and/or topical composition.

The plant seed cells frequently have the highest potential to have or to obtain all the plant cell constituents, including any therapeutic, cosmetic or medicinal actives, of that plant. The plant seed cells that have the highest potential are those which are substantially undifferentiated. As used herein, the term “substantially undifferentiated” includes seed cells that have yet to differentiate into specific plant cells, such as leaf cells, root cells, xylum and/or phloem cells, having particular functions or chemical constituents (undifferentiated seed cells); cells that have differentiated to such a limited degree that the seed cells retain a broad range of plant cell constituents that are substantially equivalent to those that are undifferentiated (near undifferentiated seed cells); and seed cells that have de-differentiated into undifferentiated seed cells (de-differentiated seed cells). In other words, the term “substantially undifferentiated” plant seed cells include undifferentiated seed cells, near undifferentiated seed cells and de-differentiated seed cells. Basically, these three types of seed cells are pluripotent and exhibit a broad range of plant cell constituents. In the present invention, the terms “botanical” and “plant” are used synonymously.

De-differentiating refers to reversing the level of differentiation in differentiated seed cells to an extent that they can be considered undifferentiated or near undifferentiated. The reversal of differentiation generally enhances the range and incidence level of plant cell constituents, including therapeutic, cosmetic and medicinal actives, within the seed cells.

Conditions that can impact de-differentiating include nutrient composition (see above), temperature, light or lack of light, gas headspace composition, operating mode and duration. Light conditions can include natural light (broad wavelength or frequency spectrum), limited or narrow wavelength or frequency spectrum, or no light (darkness). Light can also vary in intensity. Gas headspace composition can vary in oxygen, carbon dioxide and ethylene content (as well as other gases) or lack thereof. Operating mode can vary in process operation such as batch phase, semi-batch phase, continuous phase and multiple phases of the foregoing. For instance, separate phases for cell growth/proliferation and biosynthesis of actives are possible. Duration can vary according to time.

Culturing generally refers to the exposure of the plant seed cells to a suitable nutrient medium. Culturing can be carried out by adding whole seeds or portions thereof, such as the endosperm and embryo, to nutrient medium. The cells can be cultured in liquid, semisolid and solid nutrient mediums. The nutrient mediums can be formulated with salts, nutrients, hormones and elicitors to encourage cell growth, cell metabolism and proliferation, cell maintenance, biosynthesis of active ingredients or a combination of the foregoing. Examples of useful medium formulations are set forth in Table 4 of U.S. Pat. No. 6,127,181 and Examples 1 to 9 of U.S. Pat. No. 5,407,816, which examples are incorporated herein by reference.

Methods for culturing and de-differentiating plant seed cells are disclosed in U.S. Pat. Nos. 5,407,816; 5,885,826; and 6,127,181, which are incorporated in their entirety herein by reference.

Homogenizing generally refers to the breaking or fracturing of the walls of the plant seed cells. Homogenizing enhances the availability of cell seed constituents, including therapeutic, cosmetic and medicinal actives, by facilitating their release from the seed cells. Homogenizing can be carried out by mechanically fracturing the cells by any means or method known in the art or by freeze-thaw techniques also known in the art.

Preserving the plant seed cells generally refers to maintaining them under conditions such that the efficacy of desirable actives is not substantially denuded prior to incorporation into the present topical composition. Preservation is not essential to the present invention since seed cells in nutrient medium, i.e. a broth, can be directly topically applied. As a practical matter, preservation is important to ensure quality and prevent spoilage after culturing and prior to production of a topical composition on a manufacturing scale.

Preservation may be accomplished by any means known in the art such as refrigeration, storage at low pH, i.e. about 4 or less and preferably about 3.6 to about 4.0. Preferably, the seed cells are preserved by maintaining them in a nutrient broth at low pH and refrigerating them.

Advantageously, the substantially undifferentiated plant seed cells are admixed with a cosmetically acceptable vehicle to form the present composition. The cells can be separated from the nutrient medium and admixed with a vehicle, or, more preferably, the cells and/or other natural plant actives of the invention and the nutrient medium are admixed together in the vehicle. Alternately, the present composition can take the form of the cells in nutrient medium without an additional vehicle, such that the nutrient broth containing the seed cells can be directly topically applied. The broth is preferably 20 wt % plant seed cell, although broths having higher or lower plant seed cell contents are within the scope of the present invention.

Use of the active ingredients of the present invention, individually or in concert, increases the body's natural defense mechanism to stress by multiple pathways. Protection is provided to the skin, hair and nails from stress induced damage and true anti-aging benefits result through natural regenerative rejuvenation. Compositions having the active botanical ingredients of the present invention provide stress-proofing or stress-balancing benefits thus preventing stress-induced premature aging of skin, hair and nail and simultaneously providing true anti-aging skin, hair and nail care benefits.

In vitro studies have shown that compositions of the invention containing Gynostemma, Azadirachta, Rhodeola and Coconut water, either individually or in combination, provide rejuvenation benefits to the skin, hair and nails by means of a natural stress defense mechanism. More specifically, these active ingredients increase the production of stress proteins, especially heat shock proteins, in a reporter based cell assay in vitro. However, they do not increase heat shock protein levels in cells that have naturally higher levels of heat shock proteins. The actives of the present invention are also non-toxic at the amounts tested and do not induce apoptosis. The studies have shown that heat shock proteins are increased over controls in response to stress. The active ingredients up-regulate stress proteins as a natural defense to stress in response to a physiological need. In addition, Gynostemma unexpectedly produced an increase in ATP levels of cells in vitro thus producing an energizing effect. Thus, the cell can be protected through stress conditioning prior to assault, and/or the cells may be stimulated to recover from prior assault. In other words, when the invention is used in connection with, for example, skin care, the invention not only provides for treating skin which has been stressed, but as part of a daily skin care regimen, the invention also “pre-conditions” skin not to react to stressors. “Stress” is intended to include any imbalance caused by intrinsic and/or extrinsic factors, such as pollution, temperature changes, and UV radiation. Examples of stressed skin conditions include, but are not limited to, patchy skin, undereye darkness, sensitive skin and breakouts. Sensitive skin is particularly more reactive to stress and other insults.

The active ingredients of the present invention are further useful in treating, which includes preventing, arresting, ameliorating, reducing or diminishing, medical and/or cosmetic conditions of the skin, nail, lips and/or hair. Such conditions commonly include, but are not limited to, acne, psoriasis, eczema, seborrhea, dermatitis, skin and hair fragility, hair loss, hirsutism, rosacea, pruritis, calluses, warts, corns, dry skin, chapped skin, dandruff, skin blemishes, age spots, sensitive skin, hyperpigmentation or hypopigmentation, thinning skin, cellulite, stretch marks, dark circles under the eyes, freckles, yellowing, roughness, keratosis, inflammation, discoloration, skin atrophy, wrinkles, lines, hyperplasia, spider veins (telangectasia), hair loss, bruising, enlarged pores, fibrosis, sunburn, dermatological aging (chronological aging, hormonal aging and/or actinic aging), viral infections, fungal infections, bacterial infections, and any combinations thereof. The active ingredients of the present invention may also be useful in enhancing the general health, vitality and appearance of the skin, nail and hair.

Topical compositions having the active ingredients can improve the aesthetic and/or cosmetic appearance of skin. Such improvements can be manifested in any of the following: reduction in dermatological signs of aging due to, for example, chronological aging, hormonal aging, and photoaging; reduction in skin fragility; reduction in pore size; prevention and/or reversal of loss of collagen and/or elastin; ameliorating the effects of estrogen imbalance; prevention of skin atrophy; prevention and/or reduction in appearance and/or depth of lines and/or wrinkles including fine lines and/or wrinkles; prevention, reduction and/or treatment of hyperpigmentation; improvement in skin tone, radiance, clarity and/or tautness; prevention, reduction, and amelioration of skin sagging; promotion of anti-oxidant activity; improvement in skin firmness, plumpness, suppleness and/or softness; improvement in procollagen and/or collagen production; improvement in skin texture and/or promotion of retexturization; improvement in skin barrier repair and/or function; improvement in appearance of skin contours; restoration of skin luster and/or brightness; minimization of dermatological signs of fatigue and stress, e.g. skin breakout and/or resistance to environmental stress, e.g. pollution and/or temperature change; replenishment of essential nutrient and/or constituents in the skin decreased by aging and/or menopause; improvement in communication among skin cells; increase in cell proliferation and/or multiplication; increase in skin cell metabolism decreased by aging and/or menopause; retardation of cellular aging; inhibition of enzymes in the skin that accelerate aging of skin cells; minimization of skin dryness and/or improvement in skin moisturization; minimization of skin discoloration, including dark eye circles; promotion and/or acceleration of cell turnover; enhancement of skin thickness; increase in skin elasticity and/or resiliency; enhancement of exfoliation, with or without the use of alpha hydroxy acids or other exfoliants; prevention and reversal of loss of glycosaminoglycans (GAG), collagen and/or elastin; improvement in microcirculation; decrease and/or prevention in cellulite formation; and reduction in acne formation.

In particular, a topical composition having the plant actives of the present invention can improve the aesthetic appearance, health and vitality of skin. Such an improvement can be manifested in at least one of the following: prevention and/or reversal of loss of collagen and/or elastin; improvement in skin texture; improvement in skin tone, clarity, and/or tautness; promotion/acceleration of cell turnover; and enhancement of skin thickness.

The topical compositions of the present invention can be formulated in any suitable product form. Such product forms include, but are not limited to, aerosol spray, cream, dispersion, emulsion, foam, gel, liquid, lotion, mousse, ointment, patch, pomade, powder, pump spray, solid, solution, stick, and towelette.

The present composition preferably includes a vehicle. A useful vehicle is one that is pharmaceutically or cosmetically acceptable for topical applications. Useful vehicles include, but are not limited to, one or more aqueous systems, glycerin, C₁₋₄ alcohols, fatty alcohols, fatty ethers, fatty esters, polyols, glycols, vegetable oils, mineral oils, liposomes, laminar lipid materials, silicone oils, water, or any combinations thereof.

In addition, the vehicle of the compositions according to the present invention can be in the form of a homogeneous phase formulation or in the form of an emulsion including, but not limited to, oil-in-water, water-in-oil and multiple including triple, phase emulsions. These emulsions can cover a broad range of consistencies including thin lotions (which can also be suitable for spray or aerosol delivery), creamy lotions, light creams and heavy creams. Other suitable topical carriers include anhydrous liquid solvents such as oil and alcohol; aqueous-based single phase liquid solvent (e.g., hydro-alcoholic solvent system); anhydrous solid and semisolid (such as gel and stick); and aqueous based gel and mousse system. Examples of vehicle systems useful in the present invention are described in the following four references all of which are incorporated in their entirety herein by reference: “Sun Products Formulary”, Cosmetics & Toiletries, vol. 105, pp. 122-139 (December 1990); “Sun Products Formulary”, Cosmetics & Toiletries, vol. 102, pp. 117-136 (March 1997); U.S. Pat. No. 4,960,764 to Figueroa et al., issued Oct. 2, 1990; and U.S. Pat. No. 4,254,105 to Fukuda et al., issued Mar. 3, 1981.

Optionally, the present topical composition may include one or more anesthetics, anti-allergenics, antifungals, antimicrobials, anti-inflammatory agents, antioxidants, antiseptics, chelating agents, colorants, depigmenting agents, emollients, emulsifiers, exfollients, film formers, fragrances, humectants, insect repellents, lubricants, moisturizers, pharmaceutical agents, photostabilizing agents, preservatives, skin protectants, skin penetration enhancers, sunscreens, stabilizers, surfactants, thickeners, viscosity modifiers, vitamins, or any combinations thereof.

The present compositions provides for products, especially cosmetic products, that improve the condition of skin, nail and/or hair. With the present invention, it is also possible to provide compositions having higher purity, and a more therapeutically specific and standardized supply of active ingredients. Moreover, the present compositions can be formulated to deliver a consistent level of an active ingredients or blend of ingredients, so that a desired cosmetic effect is achieved, especially in batch-to-batch production of commercial products.

Compositions of the present invention can conveniently be used in any cosmetic product for skin care, make-up, personal care, hair care or nail care.

The following are non-limiting examples of the present invention. Unless indicated otherwise, all proportions and percentages are by weight

### EXAMPLE 1

### Production of Heat Shock Protein in Bioassay

The effect of various substances on the production of heat shock protein in bioassay was evaluated. Heat shock 70 protein levels were measured by utilizing a commercially available assay kit-EKS-700 (StressGen Biotechnologies, Victoria, B. C., Canada). Primary keratinocyte cells were treated with test article for approximately 18 hours. The cells were then lysed. Cell lysates were run in duplicate in the provided ELISA HSP-70 immunoassay plate and incubated for 2 hours. Plates were washed and Anti-HSP-70: Biotin antibody was added. The plate was then incubated for an additional 1 hour. The plate was washed, avidin-HRP (horse radish peroxides) conjugate was added, and allowed to incubate for 1 hour. The plate was washed again. TMB (tetramethylbenzene) substrate was added and allowed to incubate for 10 minutes; followed by addition of stop solution. Plates were measured photometrically at 450 nm and Hsp70 protein was determined.

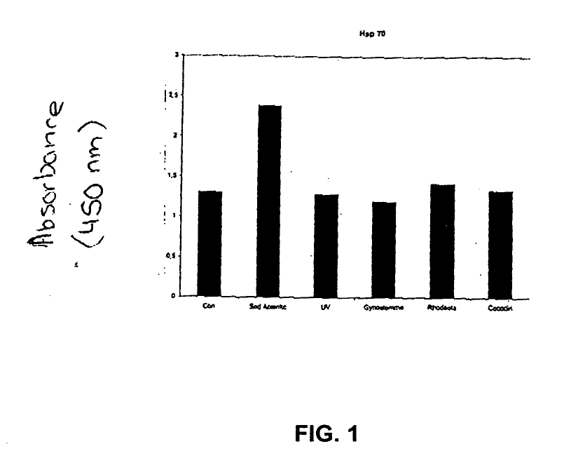

The production of heat shock protein was compared against a control of keratinocyte cells in culture medium. The following test samples were evaluated: sodium arsenite (0.001% wt./vol.+control); ultra-violet light (+control); Gynostemma (0.01% wt./vol.+control); Rhodeola (0.01% wt./vol.+control); and coconut water (0.01% wt./vol.+control). Concentrations given as % wt./vol. are based on the total weight of the ingredient per volume of the medium. Sodium arsenite is known to stimulate the production of heat shock protein. Results of this experiment are presented in FIG. 1. These data show that the botanical ingredients of the invention are non-toxic at a concentration of 0.01% wt./vol. to primary human keratinocyte cells in vitro in comparison to the heat shock protein produced by the sodium arsenite.

### EXAMPLE 2

### Apoptosis Production in Bioassay

The effect of various substances on atoptosis in bioassay was evaluated. Primary keratinocyte cells were treated with various test articles. Apoptosis is a significant event in cell physiology. During cellular stress or damage, a complex cascade process of programmed cellular death occurs, called apoptosis. Apoptosis is characterized by a series of morphologic and molecular changes described as programmed cell death. One of the earliest events in apoptosis is the activation of specific proteases called caspases.

In healthy cells, the mitochondria express two proteins Bcl-2 and Apaf-1 on their outer membrane. The Bcl-2 protein is normally bound to the protein Apaf-1. At the start of cellular distress, the Bcl-2 and Apaf-1 proteins separate. In addition, the mitochondria membrane starts to leak cytochrome C into the cytosol. The released Apaf-1 protein and cytochrome C bind to other cytosolic molecules known as caspases, specifically caspase 9. Caspases (cysteine-aspartic-acid-proteases) are widely expressed in an inactive proenzyme form in most cells. Caspase 9, cytochrome C and Apaf-1 combine to form a complex called the apoptosome. In this complex, Caspase 9 becomes active, starting a cascade of proteolytic activation of other caspases, such as caspase 8. This cascade is possible because active caspases can often activate other pro-caspases. Eventually through this cascade, caspase 3 is activated. The initial activation of caspase 3 is the major effector in protein degradation and seems to be an irreversible commitment towards cellular death.

Apoptosis (caspase 3) measurements were conducted by utilizing a commercially available assay kit (E-13183; Molecular Probes Eugene, Oreg.). Primary human keratinocytes cells were treated for 1 hour prior to lysis in lysis buffer and one “freeze-thaw” cycle. Cell lysate is centrifuged and the supernatant removed for analysis. Supernatant is added to substrate stock solution, covered, and incubated at room temperature for 30 minutes, followed my measurement with fluorescence plate reader (excitation/emission 342/441 mm). Increased fluorescence is indicative of increased caspase 3 activation.

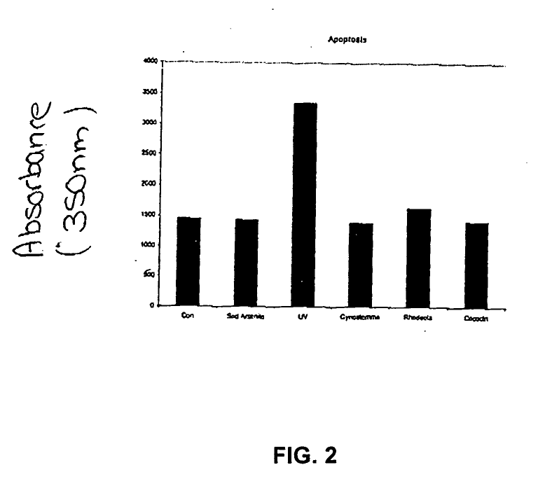

In the primary keratinocyte bioassay, the inducement of apoptosis by test substances was compared against a control of keratinocyte cells in culture medium. The following test samples were evaluated: sodium arsenite (0.001% wt./vol.+control); ultra-violet light (+control); Gynostemma (0.01% wt./vol.+control); Rhodeola (0.01% wt./vol.+control); and Coconut water (0.01% wt./vol.+control). Concentrations are given as % wt of botanical ingredient based on the total volume of the medium. Ultra violet radiation is known to induce apoptosis. Results of this experiment are presented in FIG. 2. These data show that botanical ingredients of the invention are non-toxic at a concentration of 0.01% wt./vol. to primary human keratinocyte cells in vitro as determined by inducement of apoptosis when compared to apoptosis induced by ultra violet radiation.

### EXAMPLE 3

### 25 ATP Production in Bioassay

The effect of Gynostesmma on the production of ATP in a cell culture bioassay was evaluated. Cellular ATP levels were measured by utilizing a commercially available kit (ATPLite-M) which is avaiable from Packard Bioscience, Meriden, Conn. Primary human keratinocytes were treated for 18 hours with test material or medium alone. Cells were lysed in lysis solution and placed into a 96-well microplate. Substrate solution (luciferin) was added and the plate was shaken for one minute. The plate was “dark” adapted for 10 minutes by placing in a light-tight box, followed by luminometer measurements. The greater the amount of luminescence measured, the greater the level of ATP.

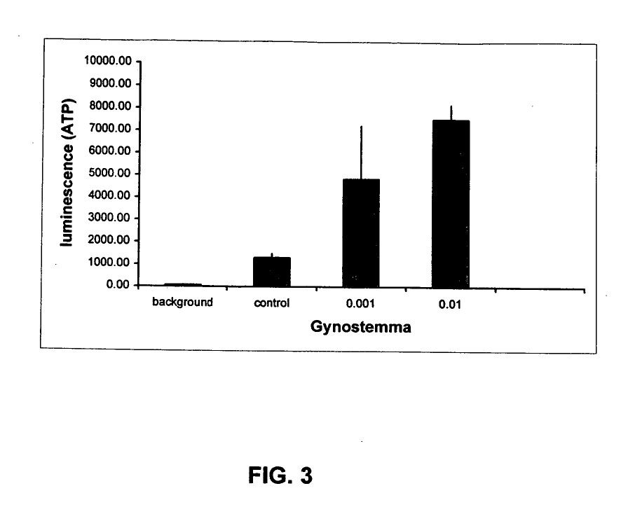

Gynostemma at concentrations of 0.001% wt./vol. and 0.01% wt./vol of botanical ingredient based upon the total volume of the medium were tested in a human keratinocyte cell culture bioassay and compared against a control of keratinocyte cells in culture medium. Background luminescence was determined. Results of this experiment are presented in FIG. 3. These data show that Gynostemma at amounts of 0.001% wt./vol and 0.01% wt/vol. significantly increase ATP production in human keratinocyte cells in vitro in comparison to control cultures.

### EXAMPLE 4

### Heat Shock Protein 70 Reporter Activation in Bioassay

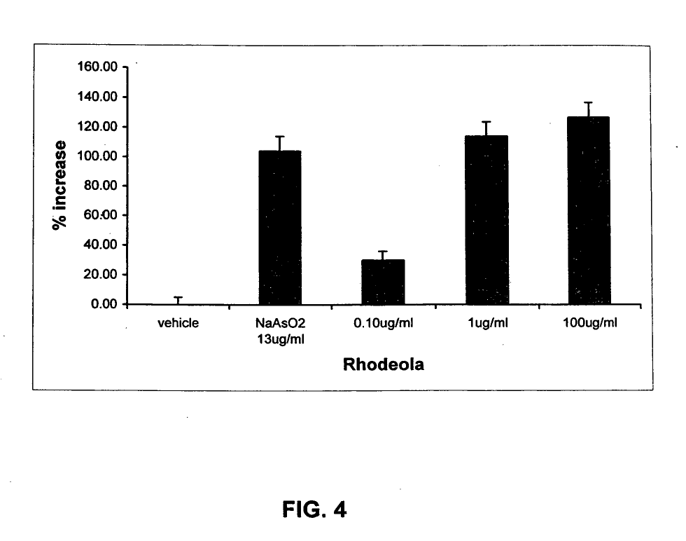

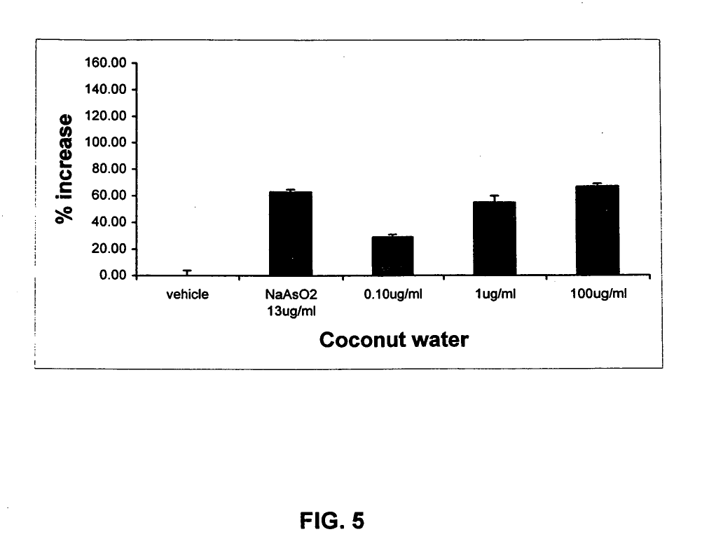

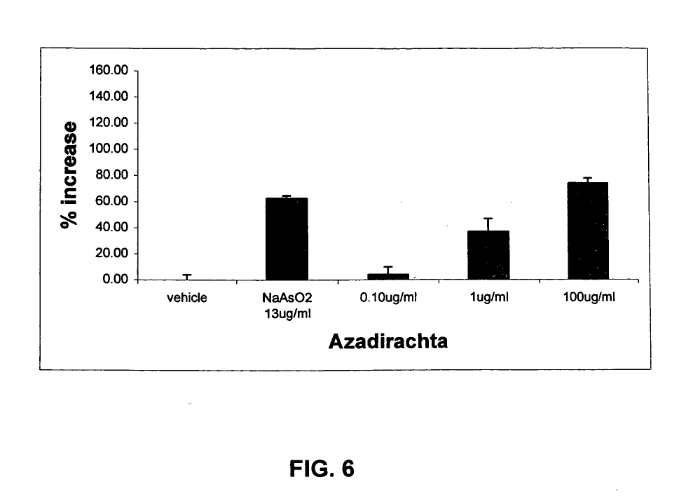

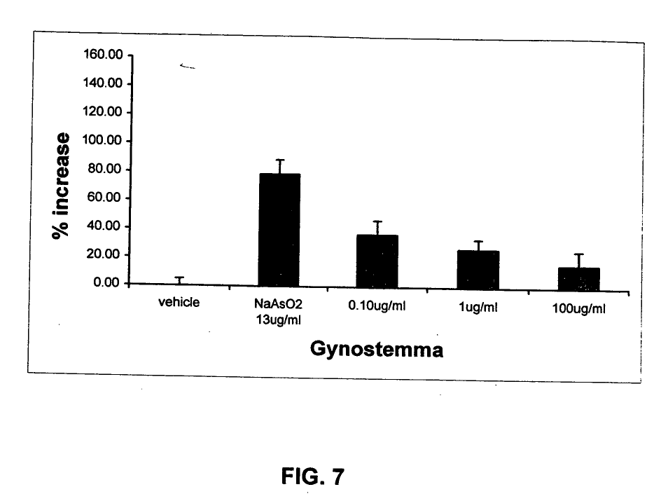

The effect of heat shock 70 protein (Hsp70) promotor activation by compositions of the invention was evaluated in a primary human keratinocyte cell culture bioassay. Primary human keratinocyte cells were transfected by electroporation with a plasmid (pCMV/Bsd) reporter system containing the Hsp70 promotor linked to a gene encoding a luciferase enzyme which acts as the reporter. Following transfection, cells containing the plasmid were selected by exposure to blasticidin. Transfected cells are resistant to blasticidin. Keratinocyte cells not transfected are killed. Resistance to blasticidin is confirmation of transfection and expression of the blasticidin deaminase gene found within the plasmid. Transfected keratinocyte cells were then treated with test materials for 24 hours. Keratinocte cell cultures were tested against NaAsO₂ (13 μg/ml), Rhodeola (0.10 μg/ml; 1.0 μg/ml; and 100 μg/ml), Coconut water (0.10 μg/ml; 1.0 μg/ml; and 100 μg/ml), Azadiracta (0.10 μg/ml; 1.0 μg/ml; and 100 μg/ml), and Gynostemma (0.10 μg/ml; 1.0 μg/ml; and 100 μg/ml). Cells were then washed and exposed to luciferin. Luciferase enzyme transcribed through activation of the Hsp70 promotor and subsequently translated into enzymatically active protein reacts with the substrate luciferin. Luciferase protein oxidizes luciferin to produce light energy that is measured by luminometer. The greater the amount of luminescence measured the greater is the activation of Hsp70 promotor. The detection of the Hsp70 promotor and light detection from luciferase activity is indicative of the production of heat shock 70 protein. Keratinocte cells in culture medium served as a control (vehicle). Results of this experiment are presented in FIG. 4 (Rhodeola), FIG. 5 (coconut water), FIG. 6 (Azadiracta), and FIG. 7 (Gynostemma). These data clearly show that the botanical ingredients of the present invention significantly cause the production of heat shock protein 70 in keratinocyte cell cultures.

It should be understood that the foregoing description is only illustrative of the present invention. Various alternatives and modifications can be devised by those skilled in the art without departing from the invention. Accordingly, the present invention is intended to encompass all such alternatives, modifications and variances that fall within the scope of the following claims.

1. A topical composition, comprising:
at least one plant ingredient selected from the group consisting of Gynostemma, coconut water, Azadiracta, and Rhodeola; and
a pharmaceutically or cosmetically acceptable vehicle, wherein when the plant ingredient is from Azadiracta seed, the composition further includes at least one other ingredient selected from the group consisting of Gynostemma, coconut water and Rhodeola.

2. The composition of claim 1, wherein the at least one plant ingredient is present in an amount about 0.0001 wt % to about 50 wt % of the total weight of the composition.

3. The composition of claim 1, wherein the at least one plant ingredient is present in an amount about 0.001 wt % to about 10 wt % of the total weight of the composition.

4. The composition of claim 1, wherein the at least one plant ingredient is present in an amount about 0.01 wt % to about 5 wt % of the total weight of the composition.

5. The composition of claim 1, wherein the at least one plant ingredient is present in an amount about 0.1 wt % to about 3 wt % of the total weight of the composition.

6. The composition of claim 1, wherein the at least one plant ingredient is Gynostemma.

7. The composition of claim 6, wherein Gynostemma is present in an amount about 0.001 wt % to about 10 wt % of the total weight of the composition.

8. The composition of claim 1, wherein the at least one plant ingredient is coconut water.

9. The composition of claim 8, wherein the coconut water is present in an amount about 0.001 wt % to about 10 wt % of the total weight of the composition.

10. The composition of claim 1, wherein the at least one plant ingredient is neem seed cell culture or its extract present in an amount about 0.001 wt % to about 10 wt % of the total weight of the composition.

11. The composition of claim 1, wherein the at least one plant ingredient is Rhodeola.

12. The composition of claim 11, wherein Rhodeola is present in an amount about 0.001 wt % to about 10 wt % of the total weight of the composition.

13. The composition of claim 1, wherein said plant ingredients are Gynostemma, coconut water, Azadirachta and Rhodeola.

14. The composition of claim 1, wherein the vehicle is one or more ingredients selected from the group consisting of aqueous systems, glycerin, C₁₋₄ alcohols, fatty alcohols, fatty ethers, fatty esters, polyols, glycols, vegetable oils, mineral oils, liposomes, laminar lipid materials, silicone oils, water, and any combinations thereof.

15. The composition of claim 1, wherein the composition is in a product form selected from the group consisting of an aerosol spray, cream, emulsion, solid, liquid, dispersion, foam, gel, lotion, mousse, ointment, powder, patch, pomade, solution, pump spray, stick, and towelette.

16. The composition of claim 1, wherein the plant ingredient is selected from the group consisting of Gynostemma, coconut water, and Rhodeola.

17. A method of improving the aesthetic appearance of skin, hair and/or nails, comprising topically applying to the skin, hair and/or nail a cosmetically effective amount of the composition of claim 1.

18. The method of claim 17, wherein the improvement is at least one improvement selected from the group consisting of reduction in dermatological signs of chronological aging, hormonal aging and/or photo-aging; reduction in skin fragility; reduction in pore size; prevention and/or reversal of loss of collagen and/or elastin; ameliorating the effects of estrogen imbalance; prevention of skin atrophy; prevention and/or reduction in appearance of lines and/or wrinkles; prevention, reduction and/or treatment of hyperpigmentation; improvement in skin tone, radiance, clarity and/or tautness; prevention, reduction, and/or amelioration of skin sagging; promotion of anti-oxidant activity; improvement in skin firmness, plumpness, suppleness and/or softness; improvement in procollagen and/or collagen production; improvement in skin texture and/or promotion of retexturization; improvement in skin barrier repair and/or function; improvement in appearance of skin contours; restoration of skin luster and/or brightness; minimization of dermatological signs of fatigue and/or stress; resistance to environmental stress; replenishment of ingredients in the skin decreased by aging and/or menopause; improved communication among skin cells; increase in cell proliferation and/or multiplication; increase in skin cell metabolism decreased by aging and/or menopause; retardation of cellular aging; inhibition of enzymes in the skin that accelerate aging of skin cells; minimization of skin dryness and/or improvement in skin moisturization; minimization of skin discoloration; promotion and/or acceleration of cell turnover; enhancement of skin thickness; increase in skin elasticity and/or resiliency; enhancement of exfoliation; prevention and reversal of loss of glycosaminoglycans, collagen and/or elastin; improvement in microcirculation; decrease and/or prevention in cellulite formation; and reduction in acne formation; and any combinations thereof.

19. The method of claim 17, wherein the improvement is at least one improvement selected form the group consisting of: reduction in dermatological signs of chronological aging, hormonal aging and/or photo-aging; prevention and/br reduction in appearance and/or depth of lines and/or wrinkles; improvement in skin tone, radiance, clarity and/or tauntness; improvement in skin firmness, plumpness, suppleness, and/or softness; and improvement in skin texture and/or promotion of retexturization; and any combinations thereof.

20. The method of claim 17, wherein the improvement is at least one improvement selected from the group consisting of minimization of dermatological signs of fatigue and/or stress; resistance to environmental stress; resistance to skin breakouts; resistance to pollution and/or temperature changes; minimization of skin dryness and/or improvement in skin moisturization; increase in skin elasticity and/or resiliency; and ameliorating the effects on skin of pollution, skin breakouts, temperature changes and/or solar radiation; and any combinations thereof.

21. The method of claim 17, wherein said composition has Gynostemma, coconut water, Azadiracta, and Rhodeola.

22. A method of improving the aesthetic appearance of skin, hair and/or nails, comprising topically applying to the skin, hair and/or nail a cosmetically effective amount of the composition of claim 16.

23. The method of claim 22, wherein the improvement is at least one improvement selected from the group consisting of minimization of dermatological signs of fatigue and/or stress; resistance to environmental stress; resistance to skin breakouts; resistance to pollution and/or temperature changes; minimization of skin dryness and/or improvement in skin moisturization; increase in skin elasticity and/or resiliency; ameliorating the effects on skin of pollution, skin breakouts, temperature changes and/or solar radiation; and any combinations.

24. The method of claim 22, wherein the at least one plant ingredient is about 0.0001 wt % to about 50 wt % of the total weight of the composition.

25. A method of treating skin, hair and/or nail comprising topically applying to the skin, hair and/or nail the composition of claim 1 in an amount effective to prevent, ameliorate, inhibit and/or reduce signs of dermatological aging.

26. The method of claim 25, wherein the signs of aging are associated with one or more from the group consisting of fatigue, physiological stress, environmental stress, pollution, temperature changes, skin breakouts, solar radiation, and any combinations thereof.

27. The method of claim 26, wherein the signs of aging are one or more signs selected from the group consisting of fatigue, physiological stress, and environmental stress.

28. The method of claim 26, wherein the signs of aging are associated with one or more signs selected from the group consisting of pollution, skin breakouts, temperature changes, and/or solar radiation.

29. A method of treating stressed skin comprising topically applying to the skin an effective amount of the composition of claim 1.

30. A method of treating stressed skin comprising topically applying to the skin an effective amount of the composition of claim 16.
